(19) United States
(12) Patent Application Publication
(10) Pub. No.: US 2017/0067028 A1
(43) Pub. Date: Mar. 9, 2017

(54) RADIOLABELING OF ADENO ASSOCIATED VIRUS

(71) Applicants: Douglas J. Ballon, Gillette, NJ (US); Ronald G. Crystal, Ithaca, NY (US); Stephen Kaminsky, Ithaca, NY (US); Paresh Kothari, Ithaca, NY (US); Bishnu De, Ithaca, NY (US); Dolan Sondhi, Ithaca, NY (US); Shankar Vallabhajosula, Ithaca, NY (US); John Babich, Ithaca, NY (US)

(72) Inventors: Douglas J. Ballon, Gillette, NJ (US); Ronald G. Crystal, Ithaca, NY (US); Stephen Kaminsky, Ithaca, NY (US); Paresh Kothari, Ithaca, NY (US); Bishnu De, Ithaca, NY (US); Dolan Sondhi, Ithaca, NY (US); Shankar Vallabhajosula, Ithaca, NY (US); John Babich, Ithaca, NY (US)

(21) Appl. No.: 15/154,647

(22) Filed: May 13, 2016

Related U.S. Application Data
(60) Provisional application No. 62/162,067, filed on May 15, 2015.

(51) Int. Cl. C12N 7/00; A61K 51/12; G01N 33/60

(52) U.S. Cl. CPC C12N 7/00; G01N 33/60; A61K 51/1203; C12N 2750/14121

(57) ABSTRACT

Provided herein are systems and methods for radiolabeling of recombinant Adeno-Associated Virus (rAAV) with radioactive iodine. Also provided are methods for in vivo imaging and treatment using the radiolabeled rAAV.

### RELATED APPLICATION

This application claims priority under 35 U.S.C. §119(e) to U.S. Provisional Application Ser. No. 62/162,067, filed May 15, 2015, the contents of which are incorporated by reference herein in its entirety.

### FIELD OF THE INVENTION

The present disclosure is directed to systems and methods for radiolabeling of Adeno-Associated Virus.

### BACKGROUND OF THE INVENTION

Adeno-Associated Viruses (AAV) are currently used to facilitate gene therapy. AAVs have shown demonstrated promise in both preclinical disease models and in human clinical trials for several disease targets. AAV generally exhibits broad tropism and low immunogenicity, which make it an attractive vector for gene therapy.

AAV is a single stranded DNA (ssDNA) virus that contains either a positive- or negative-sensed ssDNA strand of about 4.7 kilobases in length. Multiple homologous primate AAV serotypes and numerous nonhuman primate types have been identified. The genome comprises inverted terminal repeats (ITRs) (145 bases each) which can form a hairpin at each end of the DNA strand, and two open reading frames, rep and cap. The first gene encodes four proteins necessary for genome replication (Rep78, Rep68, Rep52, and Rep40), and the second expresses three structural proteins (VP-1, VP-2 and VP-3; MW 87, 72 and 62 kiloDaltons, respectively) that assemble to form the viral capsid having icosahedral symmetry. With regard to gene therapy, ITRs are required to be in cis next to the therapeutic gene while structural (cap) and packaging (rep) proteins can be delivered in trans, though a cis-acting Rep-dependent element (CARE) inside the coding sequence of the rep gene has been shown to augment the replication and encapsidation when present in cis.

AAV is typically dependent upon the presence of a helper virus, such as an adenovirus or herpesvirus, for active replication. In the absence of a helper, it establishes a latent state in which its genome is maintained episomally or integrated into the host chromosome. Packaging cell lines and helper constructs have been developed to facilitate production of AAV for gene therapy with the need for helper virus.

Gene therapy vectors using AAV can infect both dividing and quiescent cells and persist in an extrachromosomal state without integrating into the genome of the host cell, although in the native virus some integration of virally carried genes into the host genome does occur. To date, AAV vectors have been used in over 117 clinical trials worldwide. (Approximately 5.6%). Recently, promising results have been obtained from Phase 1 and Phase 2 trials for a number of diseases, including Leber's Congenital Amaurosis, hemophilia, congestive heart failure, spinal muscular atrophy, lipoprotein lipase deficiency, and Parkinson's disease

Understanding the distribution of AAV within a subject requires the sacrifice of the subject or invasive excision of tissue. In addition, the AAV genome has a small packaging size of about 4.5 kb which limits incorporation of tracking moieties in addition to the therapeutic gene.

### SUMMARY OF THE INVENTION

Provided herein, in certain embodiments, are methods for radiolabeling capsid proteins of infectious recombinant adeno-associated virus (rAAV) virions.

Described herein, in certain embodiments are methods for producing a recombinant adeno-associated virus (rAAV) labeled with radioactive iodine comprising contacting a composition containing rAAV particles with activated radiolabeled iodine to form a mixture and incubating the mixture at about 4-5° C. for at least 10 minutes. In some embodiments, the method comprises cooling the activated radiolabeled iodine solution to about 4-5° C. prior to contacting the rAAV particles. In some embodiments, the activated radiolabeled iodine is selected from among ¹²³I, ¹²⁴I, ¹²⁵I, and ¹³¹I. In some embodiments, the mixture containing the rAAV particles and activated radiolabeled iodine is incubated at about 4-5° C. for at least 20 minutes, at least 30 minutes, or at least an hour. In some embodiments, activated radiolabeled iodine activated radiolabeled iodine is generated by contacting radiolabeled iodine with iodogen (1,3,4,6-tetrachloro-3a,6a-diphenyl glycoluril) at room temperature. In some embodiments, the radiolabeled iodine is incubated with iodogen from at least 10 minutes to about 30 minutes. In some embodiments, the virus particles are concentrated prior to contacting with the solution of activated radioactive iodine.

In some embodiments, the methods further comprise purifying the radiolabeled AAV following labeling. In some embodiments, the method further comprises purifying the radiolabeled AAV following labeling using ion exchange chromatography. In some embodiments, the method further comprises purifying the radiolabeled AAV following labeling using an anion exchange cartridge. In some embodiments, the method further comprises purifying the radiolabeled AAV following incubation using an size exclusion filter. In some embodiments, the size exclusion filter has a pore size of about 80-200 Kd. In some embodiments, the size exclusion filter has a pore size of about 100 Kd. In some embodiments, the method further comprises sterilizing the radiolabeled rAAV particles. In some embodiments, the method of sterilizing comprises passing the radiolabeled rAAV particles through a 0.2 or 0.22 μm filter. In some embodiments, the rAAV encodes one or more therapeutic genes. In some embodiments, the rAAV one or more therapeutic genes are selected from the group consisting of an enzyme, a co-factor, a cytokine, an antibody, a growth factor, a hormone and an anti-inflammatory protein. In some embodiments, the rAAV rAAV encodes hCLN2. In some embodiments the serotype of the AAV particle is selected from: AAV1, AAV2, AAV3, AAV4, AAV5, AAV6, AAV7, AAV8, AAV9, AAVrh.1, rh.39, rh.43, and CSp3. In some embodiments, the rAAV is AAVrh.10 serotype. In some embodiments, the rAAV is AAVrh.10-CAG-hCLN2.

Described herein, in certain embodiments are methods for imaging an adeno-associated virus in a patient comprising, administering the radiolabeled rAAV particles prepared as described herein to a patient and detecting the virus in the patient by positron emission tomography (PET). In some embodiments, the rAAV encodes hCLN2. In some embodiments the serotype of the rAAV particle is selected from: AAV1, AAV2, AAV3, AAV4, AAV5, AAV6, AAV7, AAV8, AAV9, AAVrh.1, rh.39, rh.43, and CSp3. In some embodiments, the rAAV is an AAVrh.10 serotype. In some embodiments, the rAAV is AAVrh.10-CAG-hCLN2. In some embodiments, about 1×10¹⁰ to 1×10¹² virus particles are administered. In some embodiments, about 6×10¹⁰ rAAV particles are administered. In some embodiments, about 2 μCurie activity of rAAV is administered.

Described herein, in certain embodiments are methods for the treatment of a disease or condition comprising administering a therapeutically effective amount of the radiolabeled rAAV particles prepared as described herein to a patient in need thereof. In some embodiments the serotype of the AAV particle is selected from: AAV1, AAV2, AAV3, AAV4, AAV5, AAV6, AAV7, AAV8, AAV9, AAVrh.1, rh.39, rh.43, and CSp3. In some embodiments, the rAAV is an AAVrh.10 serotype. In some embodiments, the patient has a mutation in the CLN2 gene. In some embodiments, the rAAV encodes hCLN2. In some embodiments, the rAAV is AAVrh.10-CAG-hCLN2. In some embodiments, about 1×10¹⁰ to 1×10¹² virus particles are administered. In some embodiments, about 6×10¹⁰ rAAV particles are administered. In some embodiments, about 2 μCurie activity of rAAV is administered.

### BRIEF DESCRIPTION OF THE DRAWINGS

FIG. 1 illustrates an MRI of a human brain for assessment of vector.

FIG. 2 illustrates a section of a brain stained for TPP-1 after administration of AAVrh.10CLN2.

FIG. 3 illustrates the AAVrh.10-CAG-hCLN2 capsid labeled with Iodine 124.

FIG. 4 illustrates a graph of TPP-1 activity of radiolabeled AAVrh.10hCLN2 vector in vitro as compared to a mock.

FIG. 5 illustrates tracking by PET imaging of Iodine 124 labeled AAVrh.10CLN2 in a subject as compared to Iodine 124 unattached to AAV.

FIG. 6 illustrates tracking by PET imaging of Iodine 124 labeled AAVrh.10CLN2 in the brain of a subject.

### DETAILED DESCRIPTION OF THE INVENTION

### Certain Terminology

A “rAAV-transgene vector/virus” or “rAAV gene therapy vector/virus” refer to a recombinant adeno-associated virus (AAV) vector which is derived from the wild type AAV using molecular methods. A rAAV-transgene vector is distinguished from a wild type (wt)AAV vector, since all or a part of the viral genome has been replaced with at least one transgene, which is a non-native nucleic acid with respect to the AAV nucleic acid sequence as further described herein.

Wild type AAV belongs to the genus Dependoparvovirus, which in turn belongs to the family Parvoviridae and the subfamily Parvovirinae, also referred to as parvoviruses, which are capable of infecting vertebrates. Parvovirinae belong to family of small DNA animal viruses, i.e. the Parvoviridae family. As can be deduced from the name of their genus, members of the Dependoparvovirus are unique in that they usually require coinfection with a helper virus such as adenovirus or herpes virus for productive infection in cell culture. The genus Dependovirus includes AAV, which normally infects humans, and related viruses that infect other warm-blooded animals (e.g., bovine, canine, equine, and ovine adeno-associated viruses). Further information on parvoviruses and other members of the Parvoviridae is described in Kenneth I. Berns, “Parvoviridae: The Viruses and Their Replication,” Chapter 69 in Fields Virology (3d Ed. 1996). For convenience, the present compositions and methods are further exemplified and described herein by reference to AAV. It is, however, understood that the methods are not limited to AAV but can equally be applied to other parvoviruses.

The genomic organization of all known AAV serotypes is very similar. The genome of AAV is a linear, single-stranded DNA molecule that is less than about 5,000 nucleotides (nt) in length. Inverted terminal repeats (ITRs) flank the unique coding nucleotide sequences for the non-structural replication (Rep) proteins and the structural (VP) proteins. The VP proteins (VP 1, -2 and -3) form the capsid or protein shell. The terminal 145 nt are self-complementary and are organized so that an energetically stable intramolecular duplex forming a T-shaped hairpin can be formed. These hairpin structures function as an origin for viral DNA replication, serving as primers for the cellular DNA polymerase complex. Following wtAAV infection in mammalian cells the Rep genes 25 (i.e. Rep78 and Rep52) are expressed from the P5 promoter and the PI 9 promoter, respectively and both Rep proteins have a function in the replication of the viral genome. A splicing event in the Rep ORF results in the expression of actually four Rep proteins (i.e. Rep78, Rep68, Rep52 and Rep40). However, it has been shown that the unspliced mRNA, encoding Rep78 and Rep52 proteins, in mammalian cells are sufficient for AAV vector production. wtAAV infection in mammalian cells relies for the capsid proteins production on a combination of alternate usage of two splice acceptor sites and the suboptimal utilization of an ACG initiation codon for VP2.

A rAAV-transgene vector can have one or preferably all wild type AAV genes deleted, but can still comprise functional ITR nucleic acid sequences. Preferably, the rAAV-transgene vector does not comprise any nucleotide sequences encoding viral proteins, such as the rep (replication) or cap (capsid) genes of AAV. Functional ITR sequences are necessary for the replication, rescue and packaging of AAV virions. The ITR sequences can be wild type sequences or can have at least 80%, 85%, 90%>, 95%, or 100%) sequence identity with wild type sequences or can be altered by for example in insertion, mutation, deletion or substitution of nucleotides, as long as they remain functional. In this context, functionality refers to the ability to direct packaging of the genome into the capsid shell and then allow for expression in the host cell to be transduced or target cell. Typically, the inverted terminal repeats of the wild type AAV genome are retained in the rAAV-transgene vector. The ITRs can be cloned from the AAV viral genome or excised from a vector comprising the AAV ITRs. The ITR nucleotide sequences can be either ligated at either end to a transgene as defined herein using standard molecular biology techniques, or the wild type AAV sequence between the ITRs can be replaced with the desired nucleotide sequence. The rAAV-transgene vector preferably comprises at least the nucleotide sequences of the inverted terminal repeat regions (ITR) of one of the AAV serotypes, or nucleotide sequences substantially identical thereto, and at least one nucleotide sequence encoding a therapeutic protein (under control of a suitable regulatory element) inserted between the two ITRs. A rAAV genome can comprise of single stranded or double stranded (self-complementary) DNA. The single stranded nucleic acid molecule is either sense or antisense strand, as both polarities are equally capable of gene expression. The rAAV-transgene vector can further comprise a marker or reporter gene, such as a gene for example encoding an antibiotic resistance gene, a fluorescent protein (e.g., gfp) or a gene encoding a chemically, enzymatically or otherwise detectable and/or selectable product (e.g., lacZ, aph, etc.) known in the art.

The rAAV-transgene vector, including any possible combination of AAV serotype capsid and AAV genome ITRs, is produced using methods known in the art, as described in Pan et al. (J. of Virology (1999) 73: 3410-3417), Clark et al. (Human Gene Therapy (1999) 10: 1031-1039), Wang et al. (Methods Mol. Biol. (2011) 807: 361-404) and Grimm (Methods (2002) 28(2): 146-157), which are incorporated herein by reference. In short, the methods generally involve (a) the introduction of the rAAV genome construct into a host cell, (b) the introduction of an AAV helper construct into the host cell, wherein the helper construct comprises the viral functions missing from the wild type rAAV genome and (c) introducing a helper virus construct into the host cell. All functions for rAAV vector replication and packaging need to be present, to achieve replication and packaging of the rAAV genome into rAAV vectors. The introduction into the host cell can be carried out using standard molecular biology techniques and can be simultaneously or sequentially. Finally, the host cells are cultured to produce rAAV vectors and are purified using standard techniques such as CsC1 gradients (Xiao et al. 1996, J. Virol. 70: 8098- 8108). The purified rAAV vector is then ready for use in the methods. High titers of more than 10¹² particles per ml and high purity (free of detectable helper and wild type viruses) can be achieved (Clark et al. supra and Flotte et al. 1995, Gene Ther. 2: 29-37). The total size of the transgene inserted into the rAAV vector between the ITR regions is generally smaller than 5 kilobases (kb) in size.

In the context of the present disclosure, a capsid protein shell can be of a different serotype than the rAAV-transgene vector genome ITR. A rAAV-transgene vector of the invention can thus be encapsidated by a capsid protein shell, i.e. the icosahedral capsid, which comprises capsid proteins (VP1 , VP2, and/or VP3) of one AAV serotype, whereas the ITRs sequences contained in that rAAV-transgene vector can be from the same or different rAAV serotype.

A “serotype” is traditionally defined on the basis of a lack of cross-reactivity between antibodies to one virus as compared to another virus. Such cross-reactivity differences are typically due to differences in capsid protein sequences/antigenic determinants (e.g., due to VP1, VP2, and/or VP3 sequence differences of AAV serotypes). In many cases, serology testing for neutralizing activity has yet to be performed on mutant viruses with capsid sequence modifications to determine if they are of another serotype according to the traditional definition of serotype. Accordingly, for the sake of convenience and to avoid repetition, the term “serotype” broadly refers to both serologically distinct viruses (e.g., AAV) as well as viruses (e.g., AAV) that are not serologically distinct that can be within a subgroup or variant of a given serotype.

The term “transgene” is used to refer to a non-native nucleic acid with respect to the AAV nucleic acid sequence. It is used to refer to a polynucleotide that can be introduced into a cell or organism. Transgenes include any polynucleotide, such as a gene that encodes a polypeptide or protein, a polynucleotide that is transcribed into an inhibitory polynucleotide, or a polynucleotide that is not transcribed (e.g., lacks an expression control element, such as a promoter that drives transcription). A transgene can comprise at least two nucleotide sequences each being different or encoding for different therapeutic molecules. The at least two different nucleotide sequences can be linked by an IRES (internal ribosome entry sites) element, providing a bicistronic transcript under control of a single promoter. Suitable IRES elements are described in e.g., Hsieh et al. (1995, Biochemical Biophys. Res. Commun. 214:910-917). Furthermore, the at least two different nucleotide sequences encoding for different (therapeutic) polypeptides or proteins can be linked by a viral 2A sequence to allow for efficient expression of both transgenes from a single promoter. Examples of 2A sequences include foot and mouth disease virus, equine rhinitis A virus, Thosea asigna virus and porcine tescho virus-1 (Kim et al, PLoS One (2011) 6(4): el8556). A transgene is preferably inserted within the rAAV genome or between ITR sequences as indicated above. A transgene can also be an expression construct comprising an expression regulatory element such as a promoter or transcription regulatory sequence operably linked to a coding sequence and a 3′ termination sequence. Preferably, the coding sequence within the transgene is not operably linked to a steroid inducible promoter. More preferably, the coding sequence within the transgene is not operably linked to a dexamethasone inducible promoter

In a cell having a transgene, the transgene has been introduced/transferred/transduced by rAAV “transduction” of the cell. A cell or progeny thereof into which the transgene has been introduced is referred to as a “transduced” cell. Typically, a transgene is included in progeny of the transduced cell or becomes a part of the organism that develops from the cell. Accordingly, a “transduced” cell (e.g., in a mammal, such as a cell or tissue or organ cell), means a genetic change in a cell following incorporation of an exogenous molecule, for example, a polynucleotide or protein (e.g., a transgene) into the cell. Thus, a “transduced” cell is a cell into which, or a progeny thereof in which an exogenous molecule has been introduced, for example. The cell(s) can be propagated and the introduced protein expressed, or nucleic acid transcribed.

“Transduction” refers to the transfer of a transgene into a recipient host cell by a viral vector. Transduction of a target cell by a rAAV-transgene vector of the invention leads to transfer of the transgene contained in that vector into the transduced cell. “Host cell” or “target cell” refers to the cell into which the DNA delivery takes place, such as the synoviocytes or synovial cells of an individual. AAV vectors are able to transduce both dividing and non-dividing cells.

“Gene” or “coding sequence” refers to a DNA or RNA region which “encodes” a particular protein. A coding sequence is transcribed (DNA) and translated (RNA) into a polypeptide when placed under the control of an appropriate regulatory region, such as a promoter. A gene can comprise several operably linked fragments, such as a promoter, a 5′ leader sequence, an intron, a coding sequence and a 3′nontranslated sequence, comprising a polyadenylation site or a signal sequence. A chimeric or recombinant gene is a gene not normally found in nature, such as a gene in which for example the promoter is not associated in nature with part or all of the transcribed DNA region. “Expression of a gene” refers to the process wherein a gene is transcribed into an RNA and/or translated into an active protein.

As used herein, “gene therapy” is the insertion of nucleic acid sequences (e.g., a transgene as defined herein) into an individual's cells and/or tissues to treat a disease. The transgene can be a functional mutant allele that replaces or supplements a defective one. Gene therapy also includes insertion of transgene that are inhibitory in nature, i.e., that inhibit, decrease or reduce expression, activity or function of an endogenous gene or protein, such as an undesirable or aberrant (e.g., pathogenic) gene or protein. Such transgenes can be exogenous. An exogenous molecule or sequence is understood to be molecule or sequence not normally occurring in the cell, tissue and/or individual to be treated. Both acquired and congenital diseases are amenable to gene therapy.

A “therapeutic polypeptide” or “therapeutic protein” is to be understood herein as a polypeptide or protein that can have a beneficial effect on an individual, preferably said individual is a human, more preferably said human suffers from a disease. Such therapeutic polypeptide can be selected from, but is not limited to, the group consisting of an enzyme, a co-factor, a cytokine, an antibody, a growth factor, a hormone and an anti-inflammatory protein.

A “therapeutically-effective” amount as used herein is an amount that is sufficient to alleviate (e.g., mitigate, decrease, reduce) at least one of the symptoms associated with a disease state. Alternatively stated, a “therapeutically-effective” amount is an amount that is sufficient to provide some improvement in the condition of the individual.

In addition, reference to an element by the indefinite article “a” or “an” does not exclude the possibility that more than one of the element is present, unless the context clearly requires that there be one and only one of the elements. The indefinite article “a” or “an” thus usually means “at least one”.

The word “approximately” or “about” when used in association with a numerical value (approximately 10, about 10) preferably means that the value can be the given value of 10 more or less 10% of the value.

### Adeno Associated Virus Labeling Method

Described herein are compositions and methods for the radiolabeling of recombinant adeno-associated virus (rAAV). The present system and methods of radiolabeling of rAAV can evaluate the spatial distribution of therapeutic rAAV in a subject following administration. As such, it provides a non-invasive method for monitoring therapy with rAAV. In some embodiments, rAAV encodes a therapeutic gene. For example, the rAAV is employed for gene therapy to correct a genetic defect or deficiency. rAAV of various serotypes are known in the art and can be radiolabeled using the methods provided herein.

The technology disclosed herein facilitates direct radiolabeling of an AAV capsid. For example, the methods provided herein radiolabel the VP-1, VP-2 and/or VP-3 AAV capsid proteins. In some embodiments, the AAV capsid is labeled with radioactive iodine isotope, such as, for example, iodine-123 (¹²³I), iodine-124 (¹²⁴I), iodine-125 (¹²⁵I) or iodine-131 (¹³¹I). In particular embodiments, the radioactive iodine isotope is iodine-124. In some embodiments, the radioactive iodine isotope is detectable in vivo using a suitable method, for example, positron emission tomography (PET), single-photon emission computed tomography (SPECT), magnetic resonance imaging (MM), scintigraphy, gamma camera, a .beta.+detector, a .gamma.detector or combinations thereof.

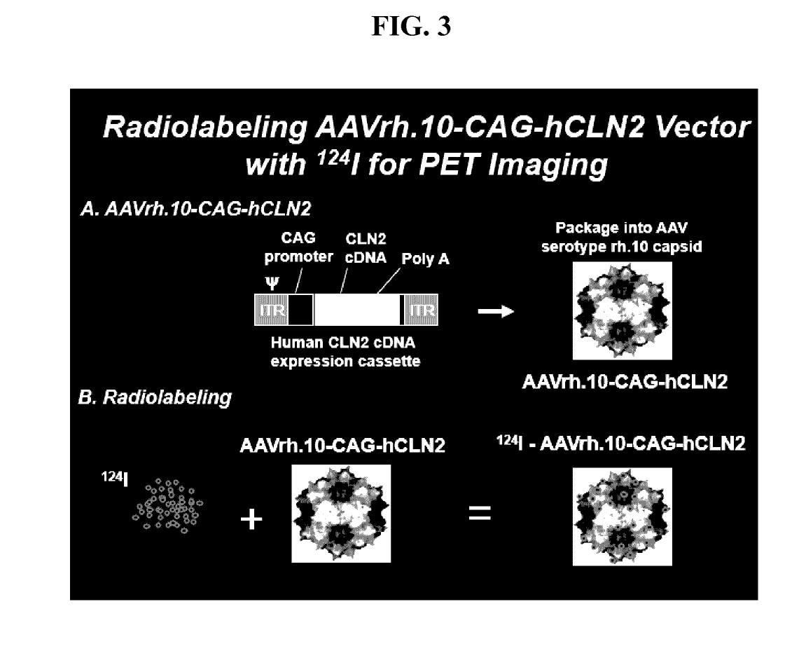

FIG. 3 illustrates an non-limiting schematic of a method for generating a radiolabeled AAV gene therapy agent. In some embodiments, a cDNA expression cassette including a promoter sequence, and an introduced gene sequence (e.g. a therapeutic gene) are packaged into an AAV capsid. FIG. 3 exemplifies a human CLN2 cDNA expression cassette packaged into an AAV serotype rh.10 capsid. However, other suitable AAV capsids and cassettes can be used. The capsid is then radiolabeled. An example of a radiolabeling method is discussed below. FIG. 3 depicts exemplary labeling of the AAVrh.10-CAG-hCLN2 capsid with Iodine 124 (¹²⁴I), though any suitable radioactive iodine isotope can be employed.

Iodination involves the introduction of the radioactive iodine into certain amino acids (usually tyrosines) present in the capsid proteins of the rAAV capsid. Iodination takes place at the positions ortho to the hydroxyl group on tyrosine. Mono- or di-substitution can occur. Radioactive iodine is incorporated into the capsid proteins in the present methods by chemical oxidation. In the chemical oxidation method, sodium iodide is converted to its corresponding reactive iodine form(e.g. I⁻ to I⁺ or I³⁻), which then spontaneously incorporates into tyrosyl groups. While necessary for iodine activation, oxidizing reagents, such as chloramine T and lactoperoxidase, are potentially damaging to proteins. Thus, the mild oxidation reagent iodogen (1,3,4,6-tetrachloro-3a,6a-diphenyl glycoluril) is employed. Typically, the iodogen is supplied on a solid substrate, such as a coated tube.

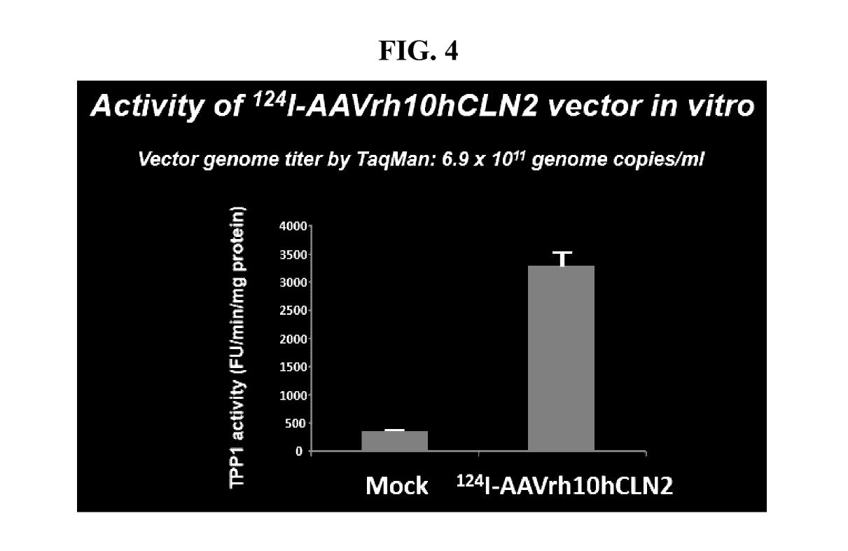

An exemplary method of radiolabeling rAAV includes the following steps: adjusting the pH of a solution of radioactive iodine (e.g. Na¹²⁴I) with a suitable iodination buffer to 7.5 or about 7.5, contacting the radioactive iodine (e.g. Na¹²⁴I) solution with iodogen (1,3,4,6-tetrachloro-3a,6a-diphenyl glycoluril) (e.g. iodogen attached to a solid substrate, e.g. an iodogen coated test tube, incubating the mixture at room temperature for about 30 min with intermittent mixing to generate activated radioactive iodine, cooling the activated radioactive iodine solution to 4-5° C., and then contacting rAAV particles with the activated radioactive iodine at 4-5° C. to radiolabel the rAAV capsids. In some embodiments, the method includes periodically mixing the solution of activated radioactive iodine and viral particles for about 1 hour during incubation at 4-5° C. Exemplary suitable iodination buffers include, but are not limited to, a Tris buffer, a phosphate buffer, or a borate buffer, optionally containing additional components, such as, for example additional salt (e.g. about 0.05-1 M NaCl). Optionally a suitable scavenging buffer can be used to remove excess unincorporated radioactive iodine. Generally, the radiolabeling does not adversely affect virus activity (see FIG. 4).

In some embodiments, the method further involves passing the product mixture through a suitable purification column, such as an anion exchange cartridge/column or immobilized metal affinity chromatography (IMAC), or ultracentrifugation. In some embodiments, the method further involves collecting the filtrate from the purification column and passing the mixture through a suitable size exclusion filter (e.g., a 80-200 Kd size exclusion filter) or by size exclusion chromotography. In particular embodiments, the filtrate is purified using a 100 Kd size exclusion filter. The desired radiolabeled virus can then be recovered from the filter and reconstituted in a suitable buffer, e.g. a phosphate buffered saline solution (PBS) or Tris buffer. Terminal filtration can also be performed through a suitable filter, such as a 0.2 or 0.22 μm filter, to render the solution sterile and suitable for administration. Exemplary purification methods for rAAV that can be used in combination with the labeling methods provided herein are also described in Burova et al. (2005) Gene Therapy 12, S5-S17.

The method described above differs from standard methods of radio iodination in several respects. For example, the iodine activation step is performed for at least 10-30 minutes prior to labeling, a cooling step is added prior to addition of the rAAV particles, and incubation is performed under the cooled conditions. It is found herein that generating the activated radioactive iodine first at room temperature and then cooling the activated radioactive iodine solution to 4-5° C. prior to contacting the viral particles increases the efficiency of radiolabeling reaction.

In some embodiments that radiolabeling procedure described herein results in a radiolabeling yield of 5%, 10%, 15%, 20%, 25%, 30%, 35%, 40%, 50%, 60%, 70%, 80%, 90% or more radiolabeled particles. In a particular, the radiolabeling procedure described herein results in a radiolabeling yield of 14.5+/−3.5% radiolabeled particles.

In some embodiments, the virus particles are concentrated prior to contacting with the solution of activated radioactive iodine (e.g. Na¹²⁴I). For example, the particles can be particles concentrated to 10⁹, 10¹⁰, 10¹¹, 10¹², 10¹³, 10¹⁴, 10¹⁵ or greater particles/ml.

In some embodiments, the iodine radioisotope for labeling is generated using a cyclotron. For example, in a non-limiting example, iodine generation includes a step of bombarding Platinum Tellurium oxide and Iodine with a proton beam. In some embodiments, the proton beam is a 13 MeV proton beam. In some embodiments, method also includes dry distillation of the radioisotope and heating in an oven with a sodium hydroxide solution. In some embodiments, heating in the oven is performed at or about 600 degrees Celsius.

### Adeno-Associated Viral Vectors

Any suitable recombinant AAV particle can labeled according the methods described herein. In some embodiments the serotype of the AAV particle is selected from: AAV1, AAV2, AAV3, AAV4, AAV5, AAV6, AAV7, AAV8, AAV9, AAVrh.1, rh.39, rh.43, and CSp3. In some embodiments, the AAV serotype is a variant of an AAV serotype is selected from: AAV1, AAV2, AAV3, AAV4, AAV5, AAV6, AAV7, AAV8, AAV9, AAVrh.10, rh.39, rh.43, and CSp3. In particular embodiments, the AAV has a capsid that is a AAVrh.10 serotype variant. In particular embodiments, the AAV particles are AAVrh.10-CAG-hCLN2 virus particles.

The radiolabeled AAV can be administered by any suitable route for delivering gene therapy, including systemically, intravenously, intraarterially, intratumorally, endoscopically, intralesionally, intramuscularly, intradermally, intraperitoneally, intravesicularly, intraarticularly, intrapleurally, percutaneously, subcutaneously, subdurally, orally, parenterally, mucosally, intranasally, intratracheally, by inhalation, intracranially, intraprostaticaly, intravitreally, topically, ocularly, vaginally and rectally.

### Exemplary Transgenes

In some embodiments, the rAAV encodes a transgene. In some embodiments, the transgene is a therapeutic gene. In some embodiments, the therapeutic gene is a normal copy of a gene that is mutated in the subject. In some embodiments, the therapeutic gene encodes a peptide inhibitor or an antagonist. In some embodiments, the therapeutic gene encodes an inhibitory RNA. In some embodiments, the therapeutic gene encodes an enzyme, a co-factor, a cytokine, an antibody, a growth factor, a hormone and an anti-inflammatory protein. In particular embodiments, the gene is CLN2.

In some embodiments, the therapeutic gene is an anti cancer gene, a tumor suppressor gene, a pro-apoptotic gene, or an anti-angiogenic gene.

In some embodiments, the therapeutic is a CNS-associated gene. In certain embodiments, the CNS-associated gene is neuronal apoptosis inhibitory protein (NAIP), nerve growth factor (NGF), glial-derived growth factor (GDNF), brain-derived growth factor (BDNF), ciliary neurotrophic factor (CNTF), tyrosine hydroxlase (TH), GTP-cyclohydrolase (GTPCH), amino acid decorboxylase (AADC) or aspartoacylase (ASPA).

### Further Detectable Genes

In some embodiments, the AAV encodes a further agent for detection, for example a detectable RNA or reporter protein. In certain embodiments, the reporter protein is a fluorescent protein, an enzyme that catalyzes a reaction yielding a detectable product, or a cell surface antigen. In certain embodiments, the enzyme is a luciferase, a beta-glucuronidase, a chloramphenicol acetyltransferase, an aminoglycoside phosphotransferase, an aminocyclitol phosphotransferase, or a Puromycin N-acetyl-transferase.

### Monitoring Gene Therapy

The technology disclosed herein can be employed to monitor gene therapy for one of a variety of diseases. In some embodiments, the short term distribution of rAAV in viral vector mediated gene therapy is examined and monitored based on the decay rate of isotope employed. Detection and imaging of the labeled virus can be effected by any suitable method, including, but not limited, to positron emission tomography (PET), single-photon emission computed tomography (SPECT), magnetic resonance imaging (MRI), scintigraphy, gamma camera, a β+ detector, a .gamma. detector and combinations thereof.

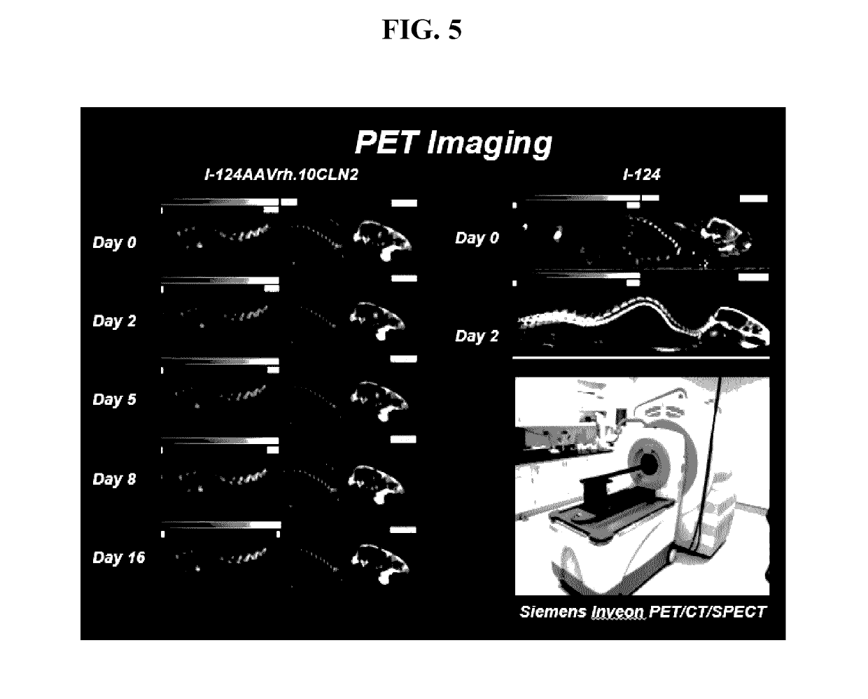

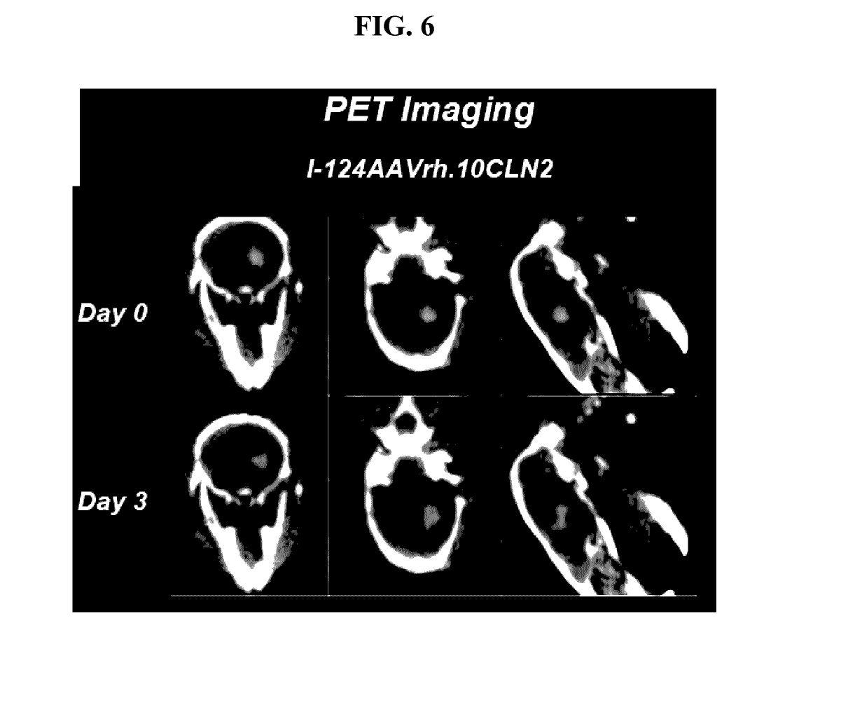

Monitoring of the rAAV virus in a subject can include delivering a suitable amount of radiolabeled AAV into the body of the subject. In some implementations, at least 1-10 μCurie activity is injected directly into the brain of a subject. In some embodiments, this activity corresponds to about 1×10¹⁰ to 1×10¹² virus particles. In some embodiments, about 6×10¹⁰ rAAV particles are administered. The injection or delivered volume can be about 1-10 microliters or another suitable volume for delivery of gene therapy and compatible with the mode of administration. The radiolabel can be imaged via positron emission tomography (PET) or another radiosensitive imaging method directly following administration and/or periodically at predetermined intervals to monitor the AAV in the subject. For example, the half-life of iodine-124 is 4.18 days and can be imaged in the subject using PET for two to three weeks. Decay of Iodine 124 can result in emission of two 511 keV photons which can be sensed by the imaging apparatus. FIG. 5 shows the tracking by PET imaging of exemplary Iodine 124 labeled AAVrh.10CLN2 in a subject as compared to Iodine 124 unattached to AAV. FIG. 6 shows tracking by PET imaging of Iodine 124 labeled AAVrh.10CLN2 in the brain of a subject.

In an exemplary embodiment, radiolabeling AAV with radioactive iodine using the methods provided herein can be employed to monitor gene therapy for diseases characterized by mutations in the CLN2 gene, which encodes tripeptidyl peptidase (TPP-I), a lysosomal protease. CLN2 disease (also called Batten Disease or Late infantile neuronal ceroid lipofuscinosis (LINCL)) is a uniformly fatal, autosomal recessive, neurodegenerative disease. The mutations in the CLN2 gene causes a deficiency in TPP-I resulting in neurons that cannot break down products of metabolism (e.g. waste membrane proteins), and eventually die. The disease onset is typically between ages 2-4. The disease results in cognitive impairment, visual failure, seizures, and deteriorating motor development, leading to a vegetative state and death by ages 8-12. Prior studies have demonstrated high level, long term TPP-I expression in the brain following intracranial gene transfer using an AAV2-based vector expressing the human CLN2 cDNA. Persistent expression CLN2 via AVV can produce sufficient amounts of TPP-I to prevent further loss of neurons, and hence limit disease progression. Exemplary CLN2 mutations associated with CLN2 disease include, but are not limited to T3016A, G3085A, G3556C, C3670T, T4383C, T4396G, and CLN2 mutations as described in, e.g., Sondhi et al. (2001) Arch. Neurol. 58, 1793-1798.

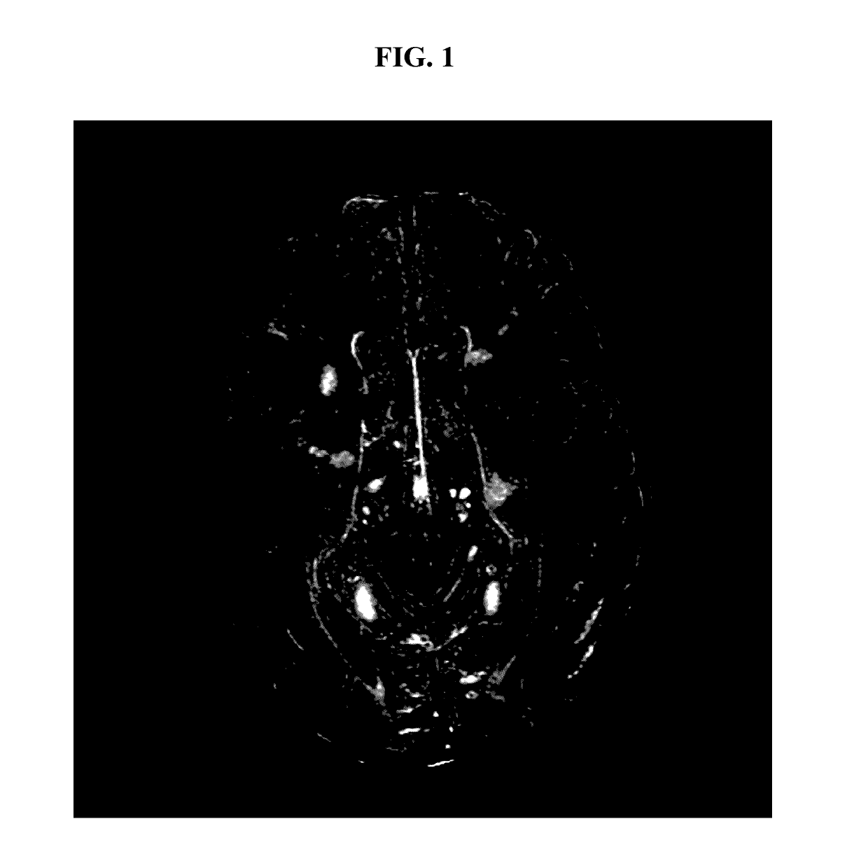

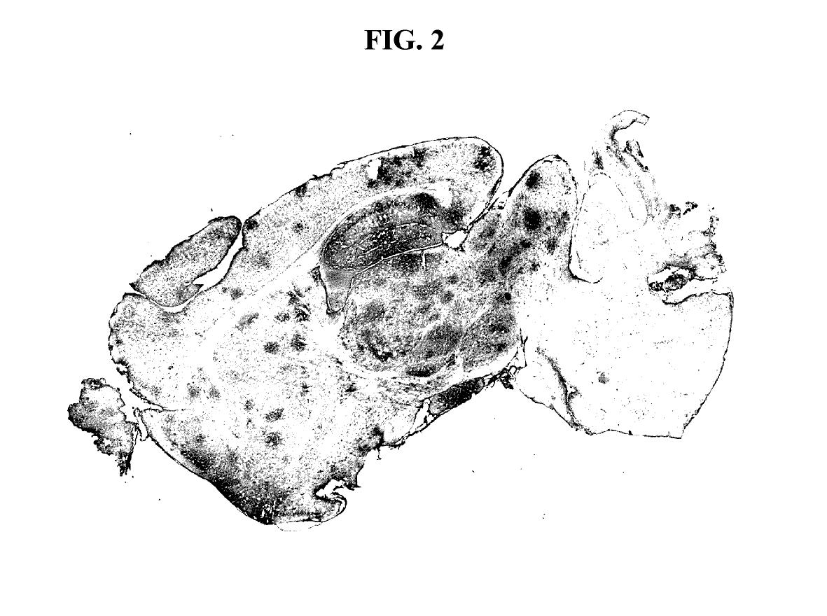

Currently, virus vector deposition in the human brain can be estimated by MRI after administration of the gene therapy. An MM of a human brain for such assessment of vector deposition of an exemplary vector, AAVrh.10CLN2, is shown in FIG. 1. In addition, excised tissue from the brain can be stained using a TPP-1 sensitive dye and analyzed ex vivo. FIG. 2 illustrates a section of a murine brain stained for TPP-1 after administration of AAVrh.10CLN2.

In particular embodiments, viral vector mediated gene therapy can be examined for CLN2 disease (LINCL, Batten disease) in the brain of a subject.

### Co-Administration

The labeled AAV for gene therapy can be administered with an additional therapeutic agent. The additional therapeutic agent can be administered before or after or simultaneously or intermittently with the virus. Additional therapeutic agents include, but not limited to, immunosuppressant, a cytokine, a chemokine, a growth factor, a photosensitizing agent, a toxin, an anti-cancer antibiotic, a chemotherapeutic compound, a radionuclide, an angiogenesis inhibitor, a signaling modulator, an anti-metabolite, an anti-cancer vaccine, an anti-cancer oligopeptide, a mitosis inhibitor protein, an antimitotic oligopeptide, an anti-cancer antibody, an anti-cancer antibiotic, an immunotherapeutic agent, and combinations thereof. Additional therapeutic agents also include.

### EXAMPLE

Late infantile neuronal ceroid lipofuscinosis (LINCL) is caused by mutations in the CLN2 gene. These defects cause neurodegeneration resulting in death by the age of 8-12 years. One treatment for LINCL that has shown promise in animal and clinical studies is gene therapy using adeno-associated virus (AAV) as a vehicle to deliver the CLN2 gene to the brain. This is currently accomplished by direct infusion, but there is no way to measure the spatial distribution of administered vector.

Iodine-124 labeling of the viral capsid offers a means for non-invasive determination of spatial distribution using MicroPET imaging.

The production of AAVrh.10CLN2 met endotoxin, mycoplasma, sterility and transgene expression release criteria. Purified AAVrh.10CLN2 was concentrated to approximately 10¹³ gene copies/ml. Labeling with Na¹²⁴I was carried out at 2-5° C. under mild oxidizing conditions in pH 7.5 iodination buffer. Following radiolabeling, the product mixture was purified using an anion exchange cartridge and centrifugal filtration. Purified ¹²⁴I-AAVrh.10CLN2 was formulated in a pH 7.4 PBS buffer. FIG. 4 depicts a graph of TPP-1 activity of an exemplary radiolabeled AAVrh10hCLN2 vector in vitro as compared to a mock infected cells.

The sterile formulation was injected (2 μl at 2.5 μCi/μl) intraparenchymally to the striatum in the murine brain and imaged on a Siemens Inveon MicroPET scanner (n=3). Thirty minute PET scans were acquired for each mouse. For the control group, the same procedure was performed using free Na¹²⁴I (n=3).

The radiolabeling efficiency was in the range of 12-14%. PET/CT imaging clearly demonstrated the spatial distribution of vector over a ten day period, with minimal uptake in the unblocked thyroid. In contrast, free iodide was rapidly cleared from the brain within 2 days. (See FIGS. 6 and 7).

This study demonstrated that Adeno-associated virus was successfully labeled with ¹²⁴I and its distribution in the mouse brain can be monitored by PET/CT imaging. This radiolabeling approach can be employed in gene therapy protocols to monitor virus distribution.

It should be understood that although the present invention has been specifically disclosed by preferred embodiments and optional features, modification, improvement and variation of the inventions embodied therein herein disclosed can be resorted to by those skilled in the art, and that such modifications, improvements and variations are considered to be within the scope of this invention. The materials, methods, and examples provided here are representative of particular embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

The inventions illustratively described herein can suitably be practiced in the absence of any element or elements, limitation or limitations, not specifically disclosed herein. Thus, for example, the terms “comprising,” “including,” containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Additional embodiments are set forth within the following claims.

1. A method for producing a recombinant adeno-associated virus (rAAV) labeled with radioactive iodine comprising contacting a composition containing rAAV particles with activated radiolabeled iodine to form a mixture and incubating the mixture at about 4-5° C. for at least 10 minutes.

2. The method of claim 1, further comprising cooling the activated radiolabeled iodine to about 4-5° C. prior to contacting the rAAV particles.

3. The method of claim 1, wherein the activated radiolabeled iodine is selected from among ¹²³I, ¹²⁴I, ¹²⁵I, and ¹³¹I.

4. The method of claim 1, wherein the mixture is incubated at about 4-5° C. for at least 20 minutes, at least 30 minutes, or at least an hour.

5. The method of claim 1, wherein the activated radiolabeled iodine is generated by contacting radiolabeled iodine with iodogen (1,3,4,6-tetrachloro-3a,6a-diphenyl glycoluril) at room temperature.

6. The method of claim 5, wherein the radiolabeled iodine is incubated with iodogen from at least 10 minutes to about 30 minutes.

7. The method of claim 1, wherein the method further comprises purifying the radiolabeled AAV following incubation using an anion exchange cartridge.

8. The method of claim 1, wherein the method further comprises purifying the radiolabeled AAV following incubation using an size exclusion filter.

9. The method of claim 8, wherein the size exclusion filter has a pore size of about 100 Kd.

10. The method of claim 1, wherein the method further comprises sterilizing the radiolabeled rAAV particles.

11. The method of claim 10, wherein the method of sterilizing comprises passing the radiolabeled rAAV particles through a 0.2 or 0.22 μm filter.

12. The method of claim 1, wherein the AAV encodes one or more therapeutic genes.

13. The method of claim 12, wherein the one or more therapeutic genes are selected from the group consisting of an enzyme, a co-factor, a cytokine, an antibody, a growth factor, a hormone and an anti-inflammatory protein.

14. The method of claim 1, wherein the rAAV encodes hCLN2.

15. The method of claim 1, wherein the rAAV is AAVrh.10 serotype

16. The method of claim 1, wherein the rAAV is AAVrh.10-CAG-hCLN2.

17. A method for imaging an adeno-associated virus in a patient comprising, administering the radiolabeled rAAV of claim 1 to a patient and detecting the virus in the patient by positron emission tomography (PET).

18. The method of claim 17, wherein the rAAV encodes hCLN2.

19. The method of claim 17, wherein the rAAV is AAVrh.10-CAG-hCLN2.

20. (canceled)

21. The method of claim 17, wherein about 2 μCurie activity of rAAV is administered.

22.-25. (canceled)
